(19) United States
(12) Patent Application Publication
(10) Pub. No.: US 2011/0091500 A1
(43) Pub. Date: Apr. 21, 2011

(54) Inactivated Live-Attenuated Bluetongue Virus Vaccine

(75) Inventors: Baptiste Dungu, Edinburgh (GB); Beate Von Teichmann, Annlin (ZA); Ian Louw, Pretoria (ZA)

(73) Assignee: Onderstepoort Biological Products Ltd., Onderstepoort (ZA)

(21) Appl. No.: 12/988,282

(22) PCT Filed: Apr. 16, 2009
(86) PCT No.: PCT/IB2009/051590

§ 371 Date: Dec. 14, 2010

(30) Foreign Application Priority Data
Apr. 16, 2008 (ZA) 2008/03366

(51) Int. Cl. A61K 39/15; C12N 7/04; A61P 31/20

(52) U.S. Cl. 424/215.1; 435/236

(57) ABSTRACT

The invention describes the preparation of an inactivated bluetongue virus (BTV) composition, and in particular a vaccine, using an attenuated live BTV vaccine strain as masterseed. The vaccine can be administered to an animal to prevent bluetongue disease by eliciting an immune response against the bluetongue virus serotype(s) included in the composition.

### BACKGROUND OF THE INVENTION

This invention relates to a vaccine composition comprising one or more inactivated bluetongue virus (BTV) serotypes prepared from live attenuated bluetongue viruses with the addition of an adjuvant.

Bluetongue disease (BT) is an arthropod-borne viral disease of sheep and cattle, caused by one or many of the 24 known serotypes of the bluetongue virus (BTV). The virus has been recognized as an important a etiological agent of disease in sheep in South Africa, and until 1943 was believed to be restricted to Africa, south of the Sahara. The disease has since been identified in several countries outside Africa, such as Cyprus, Israel, the USA, Portugal, Pakistan, India, Italy, France, Spain, China, Malaysia, Bulgaria, Australia, Argentina and most recently Kazakhstan as well as North African countries including Morocco and Tunisia. In 2006, the disease was reported for the first time in some northern European countries (Germany, Belgium and The Netherlands) and has since spread to even more European countries. BTV commonly occurs between latitudes 35° S and 40° N, but the virus has also been detected further north at beyond 48° N in Xinjiang, China, western North America and in Kazakhstan (Dungu et al., 2004).

The factors contributing to the spread of BTV include animal migration and importation, extension in the distribution of its major vector, Culicoides imicola, involvement of novel Culicoides spp. vector(s), the ability of the virus to overwinter in the absence of adult vectors, and its persistence in healthy reservoir hosts such as cattle and some wild ruminants. The eradication of BTV from endemic regions of Africa is virtually impossible due to the role played by the widely distributed Culicoides spp. midge vectors, the multiplicity of serotypes that may circulate at any point in time, and the presence and ubiquitous distribution of reservoir species, both known and unknown. However, most indigenous breeds of sheep in sub-Saharan Africa are resistant to the disease.

Strategies for the control of BT depend on whether they are aimed at outbreaks of the disease in endemic areas or in areas where the disease is not usually present. In the latter case, the aim is usually eradication, whereas in endemic areas attempts can only be made to limit the occurrence of the disease and its economic impact through vaccination.

A BTV vaccine was initially developed in South Africa more than 50 years ago and has been improved to currently include 15 of the 24 serotypes known to occur in Southern Africa (Verwoerd & Erasmus, 2004). The current vaccine consists of live attenuated, cell-adapted, plaque-purified BTV serotypes in three pentavalent vaccines, which are administered separately at 3-week intervals.

Some concerns about the current vaccine have been raised in recent years. These include:
(1) Teratogenicity of attenuated BTV vaccine strains resulting in brain defects in the fetus when administered during the first half of gestation (hence the recommendation not to administer the vaccine during the first half of pregnancy in ewes);
(2) The risk of reassortment and recombination between attenuated and virulent strains in the field; and
(3) The risk of transmission of attenuated viruses by vector midges or their release in the environment.

The need for safer BTV vaccines has been more critical during the recent European outbreaks due to the high susceptibility to BT of sheep and cattle in the affected countries, which may result in post-vaccination reactions when the currently available BTV vaccine is used.

There is therefore a need for an alternative vaccine which does not pose the same risks described above to sheep and cattle.

### SUMMARY OF THE INVENTION

According to a first embodiment of the invention, there is provided a vaccine composition including one or more inactivated BTV serotypes from the same live attenuated serotype.

The composition may include Serotype 1, 2, 3, 4, 5, 6, 7, 8, 9, 10, 11, 12, 13, 14, 15, 16, 17, 18, 19, 20, 21, 22, 23 or 24, or a combination of any two or more of these serotypes.

The BTV may be chemically inactivated. Binary ethylenimine (BEA/BEI) and/or formaldehyde may be used to inactivate the BTV.

The composition may also include any adjuvant, such as Montanide ISA 70, ISA 206, IMS 2214 or aluminium hydroxide and saponin.

The BTV composition may be used for eliciting an immune response against the target BTV serotype(s) in a vaccinated animal. Typical animals include ruminants, such as sheep, cattle, goats and wild ungulates.

The composition may be administered to the animal as a single dose or as a first dose followed by one or more booster doses.

The composition may be formulated for intra-muscular or sub-cutaneous administration.

According to a second embodiment of the invention, there is provided a method of producing a composition substantially as described above, the method comprising the step of inactivating a live, attenuated BTV.

The method may also include an earlier step of attenuating the BTV.

According to a third embodiment of the invention, there is provided a method of preventing Bluetongue disease and/or eliciting an immune response to bluetongue virus in an animal, the method comprising administering to the animal a composition substantially as described above.

According to a fourth embodiment of the invention, there is provided a method of generating antibodies specific for a BTV antigen, the method comprising the step of administering a composition described above to an animal and recovering the antibodies.

### BRIEF DESCRIPTION OF THE FIGURES

FIG. 1: Inactivation kinetics of BTV Serotype 4, BTV Serotype 2 and BTV Serotype 8 over a 48 hour incubation period. The viruses were inactivated with BEI.

FIG. 2: Summary of ELISA results obtained on pooled serum samples of the different vaccination groups up to day 84 post-vaccination.

FIG. 3: CRI post-challenge. Each group was subdivided (FIGS. 3a-e). The first 3 animals in each group were given a booster dose on day 28 post-vaccination; and they were subsequently challenged on day 60 post-vaccination. The next 3 animals (and 4 for Group F) were not given a booster dose but challenged on day 28 post-vaccination. The last 3 animals (before the 2 controls) were vaccinated and then challenged on day 60 post-vaccination. The control animals are the last 2 in all groups' graphs.

FIG. 4: Clinical reaction index in Group B1, including the unvaccinated control.

FIG. 5: Clinical reaction index in Group B2, including the unvaccinated control.

### DETAILED DESCRIPTION OF THE INVENTION

The invention describes the preparation of an inactivated bluetongue virus (BTV) composition, and in particular a vaccine, using an attenuated live BTV vaccine strain as master seed.

The composition can be administered to an animal to elicit an immune response against the bluetongue virus serotype(s) included in the vaccine.

Non-replicating virus vaccines are considered to be safer than live attenuated vaccines, as they do not pose the same risks as described earlier in this specification. Inactivated vaccines are among the most suitable non-replicating vaccines. Up until now, it has been assumed that an inactivated vaccine has to be produced from a wild type strain in order to maintain its immunogenicity to elicit a strong immune response. However, the applicant has now found that a much better virus production yield is obtained when using an attenuated strain than when using a wild-type strain. Inactivated vaccines can be obtained from the inactivation of wild-type viruses, grown in cell culture, chemically inactivated and formulated with an appropriate adjuvant. The use of live attenuated viruses makes it easier and quicker to generate inactivated vaccines, given the fact that vaccine seed materials are readily available.

The safety and efficacy of inactivated vaccines depends on the purity, innocuity, residual inactivant and toxicity, antigen load, adjuvant selected, as well as handling and administration of the vaccine by the user. Incomplete inactivation may cause a problem with virulent wild-type viruses as they may cause disease in a vaccinated animal. Validated inactivation procedures must be followed, in particular with large production volumes to ensure complete inactivation. Inactivation kinetics must be determined for large-scale production processes.

The applicant currently manufactures a BTV vaccine comprising live attenuated viruses of 15 BTV serotypes, sold as Onderstepoort Bluetongue vaccine (Reg. No. G 0358 (Act 36/1947). The generation of each vaccine strain has been a complex process of virus passages in rodents, eggs, cell culture as well as plaque selection, and testing in sheep after each passage. Table 1 below summarizes the passage history of all BTV vaccine strains contained in the current vaccine.

TABLE 1
Attenuated BTV strains currently used in vaccine production: strain
identification, their origin and passage history.

| Virus type | Strain Identification | Origin and Passage History |
|---|---|---|
| BTV-1 | Biggarsberg/8012 RSA, 1958 | 50E 3P 4BHK |
| BTV-2 | Vryheid/5036 RSA, 1958 | 50E 3P 4BHK |
| BTV-3 | Cyprus/8231 Cyprus, 1944 | 45E 2BHK 3P 5BHK |
| BTV-4 | Theiler/79043 RSA, ~1900 | 60E 3Pa 9BHK |
| BTV-5 | Mossop/4868 RSA, 1953 | 50E 2BHK 3Pa 6BHK |
| BTV-6 | Strathene/5011 RSA, 1958 | 60E 3Pa 7BHK |
| BTV-7 | Utrecht/1504 RSA, 1955 | 60E 3Pa 7BHK |
| BTV-8 | Camp/8438 RSA, 1937 | 50E 3BHK 10 Pa 7BHK |
| BTV-9 | University Farm/2766 RSA, | 70E 2BHK pp 3BHK |
| | 1942 | 7P 6BHK |
| BTV-10 | Portugal/2627 Portugal, 1956 | E81 |
| BTV-11 | Nelspoort/4575 RSA, 1944 | 35E 3P 5BHK |
| BTV-12 | Estantia/75005 RSA, 1941 | 55E 3P 4BK |
| BTV-13 | Westlands/7238 RSA, 1959 | 45E 2BHK 3Pa 4BHK |
| BTV-14 | Kolwani/89/59 RSA, 1959 | 60E 3Pa 4BHK |
| BTV-19 | 143/76 RSA, 1976 | 29E 3Pa 3BHK |

Attenuated BTV-16 is also available: BTV-16 Pakistan/7766 Pakistan 37E 3P 2BHK 1 Vero
No. E Number of passages in eggs
No. BHK Number of passages in baby hamster kidney cells
No. Vero Number of passages in green monkey kidney cells
No. P Number of large plaque selections
No. p Number of small plaque selections
a Small plaque variant

After 2 to 3 annual immunizations, most sheep are immune to all serotypes in the vaccine.

Different inactivated BTV vaccines that were generated from corresponding live attenuated BTV vaccines are described in the examples below. These examples, however, are not to be construed as limiting in any way either the spirit or scope of the invention.

### EXAMPLES

### 1. Generation of an Inactivated BTV Vaccine from Alive Attenuated BT Vaccine Virus

Monovalent bluetongue Serotypes 2, 4 and 8 (BTV2, BTV4 and BTV8, held as master seed and seed stock by the applicant were generated as per OIE standard for live bluetongue vaccine production (Howell, 1969; OIE, 2004). Working seed antigens were prepared on BHK cells from approved seed stock material and samples were sent to QC for in-process testing. Bulk virus antigen was produced using the QC-approved working seed virus by infecting confluent BHK monolayers and incubating at 37° C. until cells showed 100% CPE. The virus culture was harvested, sampled for sterility and determination of virus titre and stored at 4° C. until in-process testing was complete. Bulk vaccine was formulated by blending different virus serotypes with a stabilizer, which was transferred for final product filling. The harvest virus antigen was partially clarified by low speed centrifugation and concentrated using Polyethylene glycol (PEG) 6000 (Barteling, 1979, Sugimura & Tanaka, 1978).
PEG clarified virus was mixed 1:1 with buffered lactose peptone (BLP) and stored at −80° C. or −20° C. The stored antigen was thawed and formulated accordingly when required.
Virus inactivation
   Clarified virus antigen was inactivated with the aziridine compound binary ethylenimine (BEI) for 48 h at 37° C. This changes the nucleic acid structure of the virus by cross-linking;
   BEI is produced by the cyclization of bromoethylamine hydrobromide (BEA) which occurs under alkaline conditions;
   2-bromoethylamine hydrobromide (BEA) is dissolved in 0.175 N NaOH to make up a 0.1M BEA (Aarthi et al, 2004);
   Samples were taken hourly over the first 6 hours of the inactivation process for titration, then at 24 and 48 hours post inactivation;
   Residual BEI was neutralized by the addition of sodium thiosulphate before discarding BEI or using inactivated antigen (use 10% of the volume of BEI used);
   Samples before and after inactivation were tested on cell culture monolayers for cytopathic effect (CPE);
   The vaccine was formulated according to pre-determined antigen: adjuvant concentrations;
   Formalin could be used in the place of BEI to inactivate the virus, and/or an additional inactivation step using formalin could also be performed (although this was not done in the present process).
      Virus Titre after Harvest

The titres obtained after harvest of the 3 different BT viruses evaluated, before and after PEG concentration, are shown in Table 2.

TABLE 2
Virus titres obtained after harvest of 3 different BT viruses,
before and after PEG concentration. Titres are given in plaque
forming units per ml (pfu/ml).

| Virus type | Pre PEG | Post PEG |
|---|---|---|
| BTV-4 | 2.5 × 10⁶ | 5.5 × 10⁷ |
| BTV-2 | 1.5 × 10⁶ | 5.0 × 10⁷ |
| BTV-8 | 2.5 × 10⁷ | 4.0 × 10⁸ |

### Inactivation Kinetics

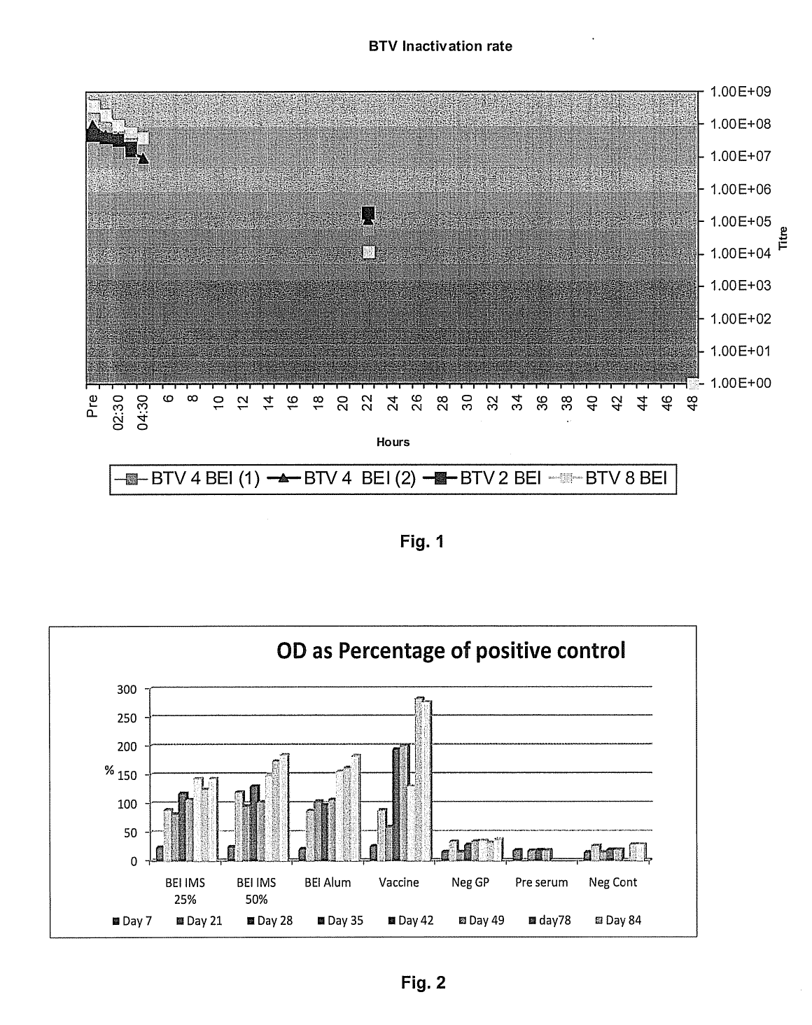

Table 3 provides an illustration of the sampling conducted during the inactivation process, with equivalent virus titre at each stage. The inactivation kinetics of the 3 viruses is summarized in FIG. 1.

TABLE 3
Inactivation kinetic of BTV-4: Samples were collected and their
virus titres determined at different stages. No samples were
collected between the 22th hour post initial inactivation and
48 hours. No pfu were detectable from 48 hours.

| Sample | Virus titre (pfu/ml) |
|---|---|
| Harvested sample | 2.5 × 10⁷ |
| Clarified by centrifugation - pre PEG | 1.5 × 10⁶ |
| PEG concentrated - pre Inactivation | 5.5 × 10⁷ |
| Supernatant after PEG concentration | 3.5 × 10⁴ |
| BEI 1 hour | 1.7 × 10⁷ |
| BEI 2:30 h | 1.4 × 10⁷ |
| BEI 3:30 h | 1.2 × 10⁷ |
| BEI 4:30 h | 6.0 × 10⁶ |
| BEI 22 h | 1.1 × 10⁴ |
| BEI 48 h | — |
| PEG Supernatant BEI 17H | — |

Inactivation with BEI results in first order kinetics inactivation, i.e. the inactivation process occurs linearly. This is in contrast with formalin inactivation that results in second order inactivation and with some viruses, incomplete inactivation. Thus, with BEI the end point can be determined. This is important for vaccines such as FMD (Foot and Mouth disease), for which there are maximum residual virus titres permissible in inactivated vaccines.

The aziridine compound binary ethylenimine (BEI) is produced by the cyclization of bromoethylamine (BEA) which occurs under alkaline conditions. The cyclization process causes a significant drop in pH from 13.5 to 8.5, which can be demonstrated visually by using the indicator B-napthol violet. This indicator was not used in this experiment. The pH values were taken manually throughout the cyclization process.

BEI is used at very low concentrations, but must nevertheless be handled with gloves as it is toxic. Residual BEI in inactivated vaccine was neutralized before use, by the addition of 0.2% of final volume of a 50% sodium thiosulphate solution.

### 2. Evaluation of the Efficacy and Safety of the Different Inactivated BTV Vaccines in Guinea Pigs and Sheep

### A) Evaluation of Inactivated BTV-4 Vaccines in Guinea Pigs

The efficacy of different formulations of inactivated BTV vaccines (iBTV-4) was evaluated in guinea pigs. Two BEI inactivated BTV-4 vaccines containing different adjuvants (aluminium hydroxide gel and Montanide IMS® adjuvant) were evaluated for immunogenicity.

### Vaccine

Inactivated monovalent BTV-4 antigen produced as described earlier, stored at −80° C., was thawed and used in two vaccine formulations, i.e. Montanide IMS (immunosol: adjuvant comprising water-based nanoparticles combined with an immunostimulant, yielding particles with sizes varying between 5 and 500 nm) from SEPPIC. France, and aluminium hydroxide, produced in-house by the applicant. The IMS was used at two concentrations: 25% and 50%.
The vaccine was formulated in such a way that a dose contained the equivalent of 10⁷ pfu/dose.
A monovalent live attenuated BTV-4 (AL-BTV4) was also produced and used together with the above 3 formulations of the inactivated BTV-4 vaccine to immunize guinea pigs.

### Animals

Four groups of 20 guinea pigs each were constituted and allocated to each of the different vaccine formulations as follows:
   Group 1: iBTV4 aluminium hydroxide;
   Group 2: iBTV4 IMS 25%;
   Group 3: iBTV4 IMS 50%; and
   Group 4: AL-BTV4.
A group of 10 unvaccinated guinea pigs was used as a control.

### Vaccination and Evaluation

All guinea pigs were pre-bled to evaluate their baseline immunological status.
Guinea pigs in Groups 1 to 4 were vaccinated on day 0 and boosted 21 days later.
Due to the difficulty in continuously bleeding guinea pigs, a schedule was established, as indicated in Table 4 below, whereby 5 guinea pigs from each group were bled on a specific day. Bleeding was weekly and conducted on days 7, 17 and 21 pre-booster and days 7, 14, 21, 28, 35, 42, 56, 63 and 70 post-booster. Different animals were bled each week, and every guinea pig was only re-bled monthly, thus giving them time to recover.
Of the control group, 2 guinea pigs were bled at each time interval.
Guinea pigs were sedated with CO₂ as analgesic and bled by cardiac puncture.
Sera from guinea pigs were tested for seroconversion using the standard neutralization assay (OIE, 2004) and AGID test. A competitive ELISA, purchased from VMRD (Veterinary Medical Research & Development, Inc.) was also used.

TABLE 4
Bleeding schedule of guinea pigs in different vaccination groups used to
evaluate different formulation of the inactivated BTV4 vaccine.

| Vaccine | | | | | | | | | | | |
|---|---|---|---|---|---|---|---|---|---|---|---|
| formulation | pre | D7 | D17 | D21 | D7 | D14 | D21 | D28 | D35 | D42 | TOTAL |
| iBTV4 Alum gel | | 5 | | | | 5 | | | | 5 | |
| | | | 5 | | | | 5 | | | | |
| | | | | 5 | | | | 5 | | | |
| | | | | | 5 | | | | 5 | | 20 |
| iBTV4 IMS 25% | | 5 | | | | 5 | | | | 5 | |
| | | | 5 | | | | 5 | | | | |
| | | | | 5 | | | | 5 | | | |
| | | | | | 5 | | | | 5 | | 20 |
| iBTV4 IMS 50% | | 5 | | | | 5 | | | | 5 | |
| | | | 5 | | | | 5 | | | | |
| | | | | 5 | | | | 5 | | | |
| | | | | | 5 | | | | 5 | | 20 |
| AL-BTV4 | | 5 | | | | 5 | | | | 5 | |
| | | | 5 | | | | 5 | | | | |
| | | | | 5 | | | | 5 | | | |
| | | | | | 5 | | | | 5 | | 20 |
| negative | 2 | | | | | 2 | | | | 2 | |
| PBS | | 2 | | | | | 2 | | | | |
| | | | 2 | | | | | 2 | | | |
| | | | | 2 | | | | | 2 | | |
| | | | | | 2 | | | | | | 10 |

### SNT Results:

Serum samples of the 5 bled GP in each group were pooled as one sample for testing. Table 5 below summarizes results obtained during the experiment.

### AGID Results:

Only day 28 post vaccination (Day 7 post booster) samples were tested using a commercial AGID test from VMRD (Inc). All samples tested were positive, except the negative control and pre serums.

### ELISA Results

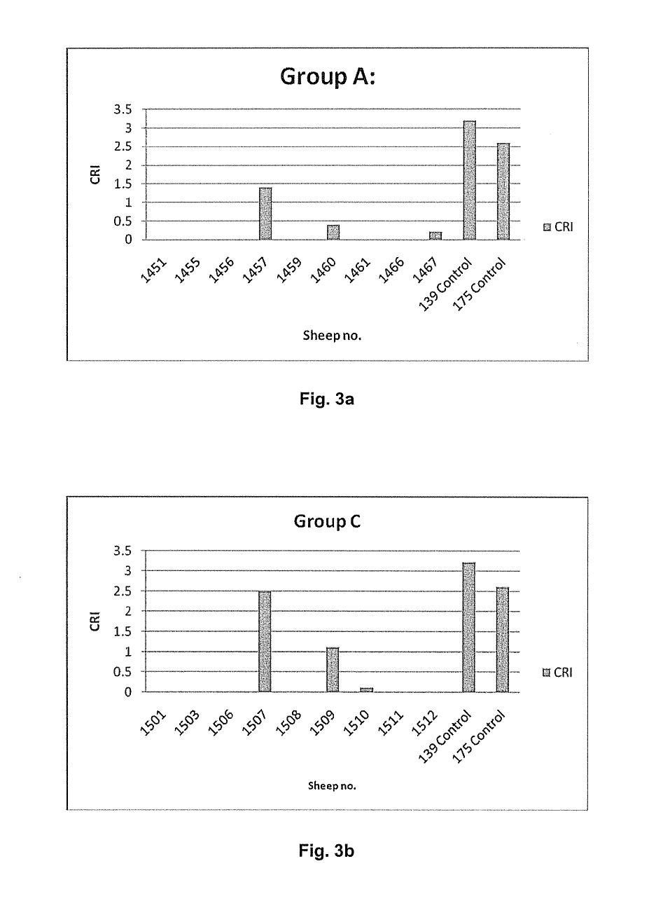

These are shown in FIG. 2.

All serological results indicated that the inactivated BTV4 vaccine was immunogenic in guinea pigs, as was the case with the live attenuated BTV4 vaccine.

Neutralizing antibodies, which are an indication of protection to BTV infection, were triggered at a protective level with all the formulations used, though with varying titres. No neutralizing antibodies were detectable before the booster dose. Similar results were also observed with the live vaccine, used here as a standard.

Detectable neutralizing antibodies were recorded from 14 days post-booster, persisting at different titres depending on the formulation. The aluminium hydroxide formulation yielded titres similar to those observed with the live vaccine.

TABLE 5
Serum neutralization test results of the pooled guinea pigs in each
vaccination group over the 84 days monitoring period.

| | | | | | | D28 | D35 | D42 | D49 | D56 | D63 | D | D70 | D77 | D84 |
|---|---|---|---|---|---|---|---|---|---|---|---|---|---|---|---|
| | | | | | | (D7 | (D14 | (D21 | (D28 | (D35 | (D42 | (D49 | (D56 | (D63 | (D70 |
| Vaccine | | | | | D21 | post | post | post | post | post | post | post | post | post | post |
| Group | Adjuvant | Pre | D7 | D14 | (Booster) | Booster) | booster) | booster) | booster) | booster) | booster) | booster) | booster) | booster) | booster) |
| BTV/BEI | Alum gel | — | — | — | ND | — | 1:64 | 1:128 | 1:32 | 1:32 | 1:32 | 1:16 | 1:8 | 1:8 | 1:8 |
| BTV/BEI | IMS 25% | — | — | — | ND | — | 1:8 | 1:32 | 1:4 | 1:4 | 1:4 | 1:4 | < | < | < |
| BTV/BEI | IMS 50% | — | — | — | ND | — | 1:64 | 1:128 | 1:8 | 1:4 | 1:4 | < | < | < | < |
| BTV4 | None | — | — | — | ND | — | 1:128 | 1:128 | 1:32 | 1:32 | 1:32 | 1:16 | 1:4 | 1:4 | 1:4 |
| OBP | | | | | | | | | | | | | | | |
| attenuated | | | | | | | | | | | | | | | |
| strain | | | | | | | | | | | | | | | |
| negative | PBS | — | — | — | | — | — | — | — | — | — | — | — | — | — |

ND—Not done

The aim of the present study was to assess the ability of the formulated inactivated BTV4 vaccine to trigger an immune response that could be correlated to protection. Since a single antigen payload was used (antigen in a dose equivalent to 10⁷ pfu/dose), it is not possible to attribute the lower SNT titres observed with the IMS 2214 formulations to poorer protection ability. Furthermore, the ability of a specific adjuvant to trigger a stronger immune response may vary between animal species: a poorer response in guineas pig may be different in sheep or cattle.

The ELISA results showed that the guinea pigs vaccinated with all different formulations seroconverted and developed increasing antibody titres. Once again, animals vaccinated with the AL-BTV4 showed higher titres than those vaccinated with the different formulations of the inactivated vaccine.

AGID tests were done only on serum from day 14 post-booster and found to be positive.

It was concluded that the inactivated BTV4 vaccine, generated from the live attenuated BTV4 vaccine, was capable of generating an immune response and protective neutralizing antibodies comparable to those generated with the live attenuated vaccine. The results also indicated the need to evaluate the vaccine in target animals, i.e. sheep, using different formulations.

### B) Evaluation of Inactivated BTV4 Vaccine in Sheep

The efficacy and safety of different formulations of the inactivated BTV4 vaccine on sheep were evaluated. The following adjuvants were used to formulate 4 different types of the iBTV4 vaccine:
Aluminium hydroxide;
Montanide ISA 70: a commercial water in oil adjuvant (SEPPIC, France);
Montanide ISA 206 a commercial water in oil in water adjuvant (SEPPIC, France);
IMS 2214 immunosol: adjuvant comprising water-based nanoparticles (SEPPIC, France); and
Aluminium hydroxide gel and saponin, produced by the applicant.

The iBTV4 antigen produced as described earlier and stored at −80° C. was used to generate the 4 different formulations under the following mixing conditions:
ISA 70: 70% w/w oil phase and 30% w/w water (antigen) phase;
ISA 206: 50% w/w of oil and water phase each;
Aluminium hydroxide gel: 12.5% final concentration of the gel;
IMS 2214: 50% w/w of the oil and water phase each;
Aluminium hydroxide gel and saponin: 12.5% final concentration of gel and 1.25 mg of saponin per vaccine dose.

Each formulation was used to immunize 5 groups of 3 sheep, according to different schedules, as described in Table 6 below. A further 16 sheep were used as controls.

TABLE 6
Vaccination and challenge schedule used to immunize sheep with 5
different formulations of the inactivated BTV4 vaccine.

| iBTV-4 | Sheep | | Vaccine | | Challenge | | Observation |
|---|---|---|---|---|---|---|---|
| vaccines | Group | No | Day 0 | Day 28 | Day 28 | Day 49 | Day 180 |
| Alum | 1A | 3 | ✓ | ✓ | | ✓ | |
| | 2A | 3 | ✓ | | ✓ | | |
| | 3A | 3 | ✓ | | | ✓ | |
| | 4A | 3 | ✓ | ✓ | | | ✓ |
| | 5A | 3 | ✓ | | | | ✓ |
| | | control | | | 1 | 1 | |
| ISA 206 | 1C | 3 | ✓ | ✓ | | ✓ | |
| | 2C | 3 | ✓ | | ✓ | | |
| | 3C | 3 | ✓ | | | ✓ | |
| | 4C | 3 | ✓ | ✓ | | | ✓ |
| | 5C | 3 | ✓ | | | | ✓ |
| | | control | | | 1 | 1 | |
| ISA 70 | 1D | 3 | ✓ | ✓ | | ✓ | |
| | 2D | 3 | ✓ | | ✓ | | |
| | 3D | 3 | ✓ | | | ✓ | |
| | 4D | 3 | ✓ | ✓ | | | ✓ |
| | 5D | 3 | ✓ | | | | ✓ |
| | | control | | | 1 | 1 | |
| IMS 2214 | 1E | 3 | ✓ | ✓ | | ✓ | |
| | 2E | 3 | ✓ | | ✓ | | |
| | 3E | 3 | ✓ | | | ✓ | |
| | 4E | 3 | ✓ | ✓ | | | ✓ |
| | 5E | 3 | ✓ | | | | ✓ |
| | | control | | | 1 | 1 | |
| Alum/Sap | 1F | 3 | ✓ | ✓ | | ✓ | |
| | 2F | 3 | ✓ | | ✓ | | |
| | 3F | 3 | ✓ | | | ✓ | |
| | 4F | 3 | ✓ | ✓ | | | ✓ |
| | 5F | 3 | ✓ | | | | ✓ |
| | | control | | | 1 | 1 | |

All sheep were pre-bled 3 days prior to commencement of the study. Rectal temperatures of the sheep were monitored and recorded twice daily from 3 days prior to the first vaccination and for 28 days post-vaccination, 21 days post booster (where applicable) and 21 days post-challenge. Sheep were bled for serum and whole blood (5 ml each) on days 0, 3, 7, 10, 14, 18, 21, 24 and 28 post-vaccination(s) and challenge day, to determine neutralising antibody responses and viremia levels. Eight sheep from each group (total of 64) were maintained for a 6 month period, in an insect-free facility. The temperatures of these sheep were also monitored, and they were bled on days −3, 0, 3, 7, 10, 14, 18, 21, 24 and 28 post-vaccination(s) for serum and whole blood; and weekly on days 35, 42, 49 and 56, and twice monthly thereafter (days 60, 74, 98, 112, 126, 140, 154, 168 and 182) for serum only.

Body temperatures and other clinical signs of the sheep were monitored daily for 21 days post-vaccination and post-challenge. Clinical reaction index (CRI) (Huismans et al., 1987; Lacetera & Ronchi, 2004) was used to quantify the observed clinical reactions. Briefly, the CRI was calculated as the sum of the following:
a. Temperature score—the cumulative total of fever readings above 40° C. on days 3 to 14 post-challenge;
b. Clinical lesion score—lesions of the mouth, nose and feet were each scored on a scale of 0-4 and added together;
c. Death—an additional 4 points were added if death occurred within 14 days post-challenge.

Serum samples were taken weekly after vaccination/challenge for evaluation of seroconversion.

### Serum Neutralization Test Results:

Table 7 below summarizes serum neutralization results obtained on sheep in different groups, monitored up to 133 days post initial vaccination.

TABLE 7
Serum neutralization test (SNT) results obtained on samples of sheep in
different vaccine formulation groups up to day 133 post initial vaccination.
Undetectable titres are represented by < sign. The light grey cells
represent vaccination while the dark grey cells represent challenge.

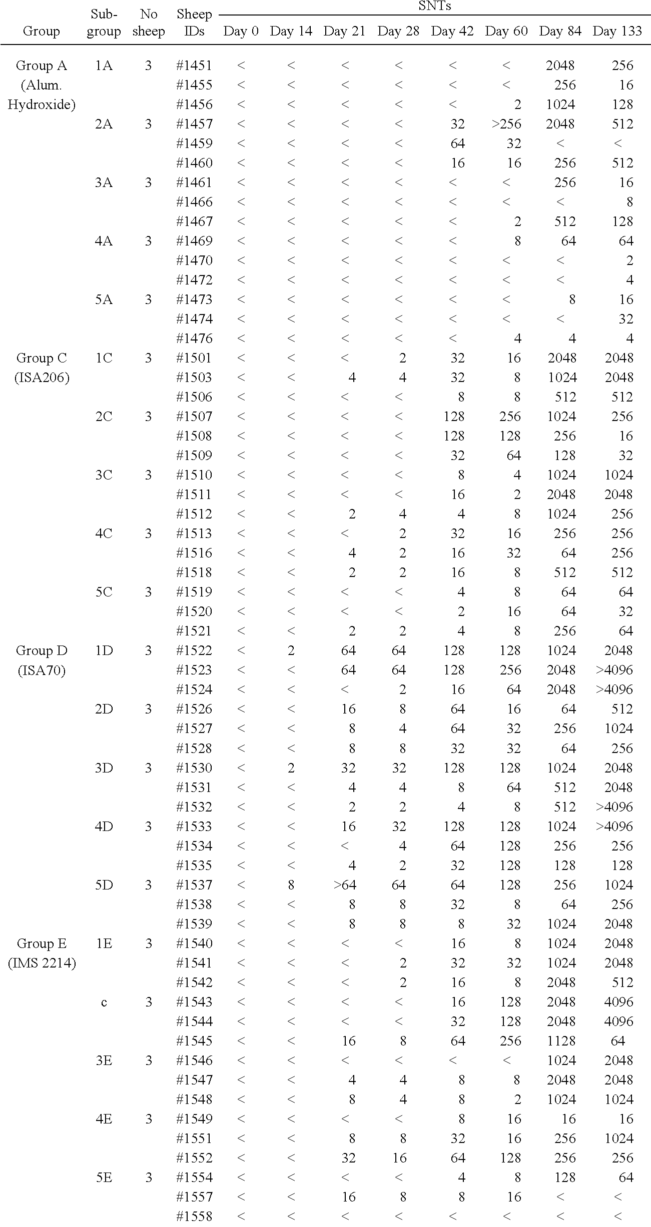

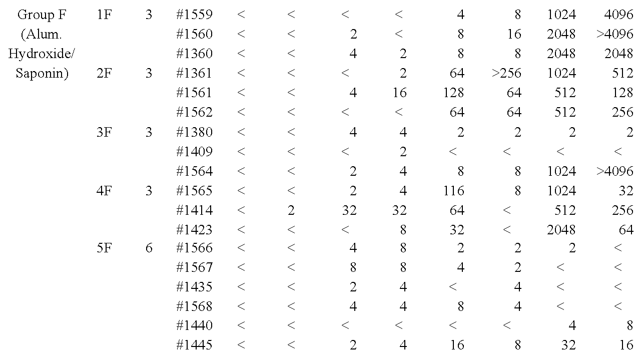

### Clinical Reaction Index Results

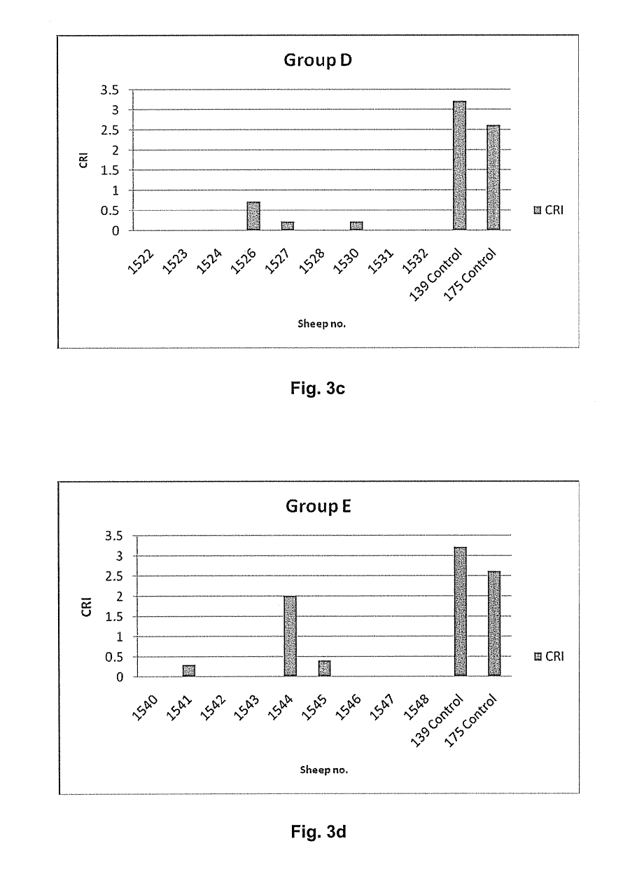

The CRI were calculated only for the 3 subgroups that were challenged, i.e. animals vaccinated twice and vaccinated 28 days post-booster, and animals vaccinated once and challenged 28 and 49 days post-vaccination, respectively. FIGS. 3a-e summarizes the CRI recorded for each animal in the different vaccination groups, together with the unvaccinated challenged controls.

The results obtained with the different formulations of the iBTV4 indicated that the adjuvant used played a role in the onset of immunity, as illustrated by SNT results and the level of protection afforded as indicated by the CRI results. Detectable SNT titres, up to 1:64, were recorded after a single vaccination in sheep immunized with the ISA 70 formulation, and to a lesser extent with the IMS 2214 and aluminium hydroxide/saponin already on day 21 post-vaccination, while no detectable titres were seen in the aluminium hydroxide group and very low titres were seen in the ISA 206 group. The booster vaccination improved the SNT response in all groups, except for the aluminium hydroxide group. The situation of sheep vaccinated with the ISA 206 formulation improved following the booster, but to a lesser extent than the other groups. Overall the ISA 70 formulation seemed to be better than all the others, following a single vaccination or two vaccinations.

Animals that were challenged after a booster dose in all of the 5 groups were protected, as illustrated by the CRI results: none of them had a CRI higher than one, while the unvaccinated positive controls had a CRI varying between 2.5 and 3.

Protection following a single vaccination was better in the ISA 70 group and to a lesser extent in the aluminum hydroxide/saponin group, thus correlating with the SNT results.

The generation of clinical signs following a virulent challenge varies enormously between different serotypes, and depends on factors such as sheep susceptibility. BTV4 generally does not result in severe clinical signs, even in more susceptible sheep breeds, as seen in the Spanish and Portuguese BTV4 outbreaks (OIE disease information, 2006). This could explain the lower CRI observed in the positive unvaccinated controls used in the present study. It was, however, still possible to see a difference between the clinical responses in vaccinated and unvaccinated animals.

The results of the present study clearly indicated that the iBTV4 vaccine was immunogenic and protective, with the adjuvant playing an important role in improving the protection afforded. The SNT results correlated positively with the CRI results, confirming the value of each of the two parameters.

### C) Evaluation of Inactivated BTV8 Vaccine in Sheep

The ability of an inactivated BTV8 vaccine to protect sheep against a virulent challenge of the European BTV8 wild-type virus was evaluated after single and booster vaccine doses, together with the safety of the vaccine. The inactivated BTV8 vaccine was based on the attenuated BT Serotype 8 vaccine virus.

Ten adult Merino ewes aged 1-2 years were used in this trial. Bluetongue (BT) susceptibility was confirmed by a competitive ELISA (OIE, 2004) in the BT reference laboratories at the Onderstepoort Veterinary Institute (OVI) of South Africa. Animals were stabled in an insect free stable for the duration of the experiment.

Inactivated monovalent BTV8 vaccine was produced as described in previous reports. Briefly, BTV8 attenuated seed stock antigen was used to infect 4 BHK roller flasks. Harvested antigen was precipitated using PEG as described earlier. Concentrated antigen was inactivated using BEI. The iBTV8 vaccine was formulated using ISA 206 as adjuvant.

Vaccinated sheep were monitored daily for 21 days post-vaccination and 21 days post-challenge for temperature and clinical signs. All sheep were exposed to environmental stresses daily for 3-4 hours post-challenge.

The Netherlands outbreak virus isolate BTV8 (G) 5/10A 2006/01KC 3BHK received from the OIE Bluetongue reference laboratory at OVI was used as challenge dose. A virus suspension of an additional passage in BHK cells with a dose of 10⁷ pfu/ml was administered by the intravenous route.

Two groups of sheep were used in this trial (Table 8). One group was vaccinated once and challenged 28 days post-vaccination. The other group was boosted 21 days after the first vaccination, and then challenged 28 days post-vaccination.

TABLE 8
Vaccination and challenge schedule of inactivated BTV8 in sheep.
Animals in Group B1 were vaccinated once and challenged 28 days later.
Animals in Group B2 were vaccinated and boosted 28 days later,
and challenged 28 days post-booster.

| | | | Booster | Challenge | |
|---|---|---|---|---|---|
| | | | Day 28 | Day 28 | Day 28 |
| | | | post- | post- | post- |
| Group | Ovine no. | Vaccine | Vaccination | Vaccination | booster |
| B1 | #66 | iBT8 | | | |
| | #67 | | | | |
| | #84 | | | | |
| | #93 | | | | |
| B2 | #95 | iBT8 | | | |
| | #97 | | | | |
| | #113 | | | | |
| | #129 | | | | |

Sheep were monitored daily for 21 days post-vaccination and challenge, for body temperature and other clinical signs. Clinical reaction index (CRI) (Huismans et al., 1987; Lacetera & Ronchi, 2004) was used to quantify the observed clinical reactions, as described earlier herein.

Vireamia in sheep was determined according to OIE guidelines (OIE, 2004). In brief, a 25 cm³ Falcon flask was inoculated with 1 ml of blood and incubated for +/−30 min at 37° C. before blood was rinsed from the flask and maintenance medium added. Negative samples were passaged a further two generations on tissue culture.

Serum samples were collected weekly after vaccination and after challenge for serological evaluation. Neutralising antibodies were determined by the microtitre serum neutralization test using serotype BTV8 neutralizing antigen (OIE, 2004) (Table 9).

TABLE 9
SNT titres of sheep in Groups B1 and B2 following vaccinations and
challenges: SNT titres in the first and second columns (day 21 and day
28) represent neutralizing antibodies after one vaccination in both Groups
B1 and B2. The third and fourth columns (day 14 post-challenge/booster)
represent neutralizing antibody titres following challenge in Group B1 (14
and 21 days post-challenge) and booster in Group B2 (14 and 21 days
post-booster). The fifth column represents neutralizing antibody titres from
samples taken 28 post challenge for Group B2, this represents day 56
days post challenge for group B1

| | | Day 21 | Day 28 | | | Day 56 |
|---|---|---|---|---|---|---|
| | | Post | Post | Day 14 post- | Day 21 post- | post- |
| Group | Ovine no. | Vaccination | Vaccination | chall | chall | challenge |
| B1 | #66 | 2 | 4 | 512 | 1024 | 512 |
| | #67 | 4 | 8 | 256 | 512 | 512 |
| | #84 | 4 | 8 | 128 | 256 | 512 |
| | #93 | 2 | 16 | 128 | 512 | 1024 |
| | | Day 21 | Day 28 | | | Day 28 |
| | | Post | Post | Day 14 post- | Day 21 post- | post- |
| | | Vaccination | Vaccination | booster | booster | challenge |
| B2 | #95 | 8 | 32 | 32 | 32 | 512 |
| | #97 | 8 | 16 | 32 | 128 | 512 |
| | #113 | 16 | 32 | 32 | 64 | 256 |
| | #129 | 8 | 16 | 32 | 32 | 256 |
| Control | #145 | <2 | <2 | 64 | 256 | >1024 |

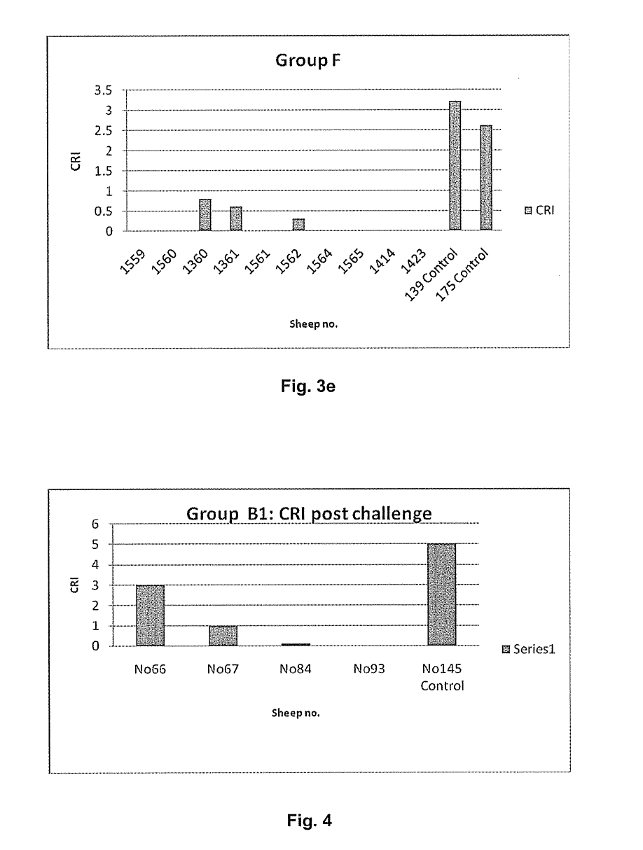

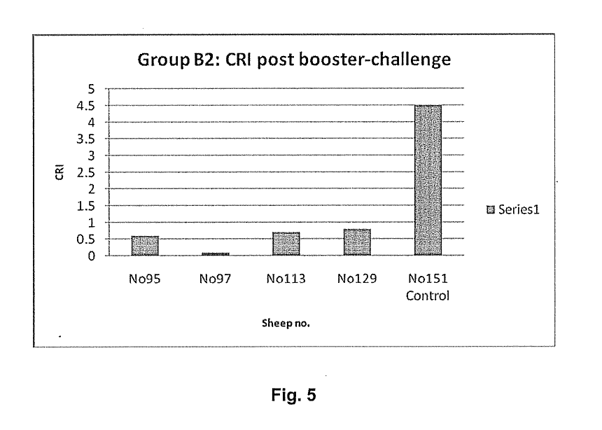

Although all vaccinated animals showed temperature reactions to differing degrees post-challenge, the sheep that received a booster dose showed less temperature reaction, and subsequently had a lower CRI than those in Group B1 (FIGS. 4 and 5). Unvaccinated control sheep, however, developed slight hyperemia of the nasal and buccal mucosa and slight erosion in the mouth, noticeable between days 5 and 9 post-challenge. These signs, combined with temperature reactions lasting a few days, resulted in CRI of 4.5 and 5. The low level of CRI in animals boosted (lower than 1) indicates that they would be protected against a virulent challenge.

Neutralizing antibody response to a single vaccination with iBTV was low (Table 9), and the highest response 28 days post-vaccination was 32. A booster inoculation (Group B2) gave results of between 32 and 128. This level of antibody should be sufficient to protect animals against challenge. SNT of Group B2 post-challenge showed a significant rise from values post-booster. Antibody response after a single vaccination and challenge showed a similar drastic increase in the antibody levels.

The limited ability of the iBTV vaccine formulated with ISA 206 to trigger a full protective immunity after one vaccination was comparable to results described above with the iBTV4 vaccine. Even though sheep vaccinated with the iBTV8 vaccine were protected against a virulent challenge with the European BTV8, and on the basis of results described in example B above, the applicant is planning to conduct a comparative trial using an ISA 70 adjuvanted iBTV8 vaccine.

### D) Formulation Procedure

The vaccine formulation of the invention can be made by formulating clarified virus with ISA70. The suspension BHK-21 cells from ASG are scaled up from spinner flasks to 1000 L to maximum cell density. The cell culture is transferred to a 2000 L bioreactor and adjusted/diluted to a start cell density of 1-1.5×10⁶ cells/ml. The cells are infected with virus and the virus culture harvested after 48 hours. The virus is clarified using a dead-end filter and titrated to determine a pre-inactivation titre. The antigen is inactivated with 10 mM BEI for 38 hours and tested to confirm inactivation and sterility. Bulk vaccine is formulated using ISA70 as an adjuvant in 30% antigen: 70% ISA70 ratio.

The applicant has thus shown that the different inactivated BTV vaccines that it generated were immunogenic in guinea pigs and sheep, and protected sheep against a virulent challenge. The adjuvant used played an important role in triggering an early and solid immunity, protecting vaccinated animals after either a single vaccination or two vaccinations.

Further studies using various inactivated BTV serotype combinations and different adjuvants are currently in progress.

While the invention has been described in detail with respect to specific embodiments thereof, it will be appreciated by those skilled in the art that various alterations, modifications and other changes may be made to the invention without departing from the spirit and scope of the present invention.

### REFERENCES

Aarthi D. Ananda Rao K, Robinson R, Srinivasan V A. (2004): Validation of binary ethyleneimine (BEI) used as an inactivant for foot and mouth disease tissue culture vaccine. Biologicals 32(3): 153-156.
Barteling S J. (1979): Some aspects of FMDV-production in growing cells and a closed system for concentration of FMDV by polyethylene glycol. Dev Biol Stand. 42: 71-74.
Dungu B, Gerdes T & Smit T (2004). The use of vaccination in the control of bluetongue in southern Africa. Vet. Italiana 40(4): 616-622.
Howell P G (1969). The antigenic classification of strains of bluetongue virus, their significance and use in prophylactic immunization. Doctoral thesis, Faculty of Veterinary Science, University of Pretoria
Huismans H, van der Walt N T, Cloete M, & Erasmus B J (1987). Isolation of a capsid protein of bluetongue virus that induces a protective immune response in sheep. Virology, 157 (1): 172-179.
Lacetera N & Ronchi B (2004). Evaluation of antibody response and nonspecific lymphocyte blastogenesis following inoculation of a live attenuated bluetongue virus vaccine in goats. Am. J. Vet. Res 65 (10): 1331-1334.
Murray P K & Eaton B T (1996). Vaccines for bluetongue. Aust. Vet. J. 73 (6): 207-210.
OIE (2004). Bluetongue. In Manual of Diagnostic Tests and Vaccines for Terrestrial Animals. Paris: Office International des Epizooties.
OIE Disease Information (2006). Bluetongue in Spain: Follow up report no. 2; Vol. 19 (3).
Sugimura T & Tanaka Y (1978). The use of polyethylene glycol in concentration and purification of several bovine viruses. Natl Inst Anim Health 18(2): 53-57.
Verwoerd D W & Erasmus B J (2004). Bluetongue. In Infectious diseases of livestock. Eds J. A. W. Coetzer & R. C. Tustin, 2^{nd} edition. Oxford University Press, Cape Town.

1. A composition comprising an inactivated bluetongue virus (BTV) prepared from a live attenuated BTV.

2. A composition according to claim 1, which includes more than one inactivated BTV.

3. A composition according to claim 1, wherein the BTV is any one or more of serotypes 1 to 24.

4. A composition according to claim 3, wherein the BTV is serotype 2.

5. A composition according to claim 3, wherein the BTV is serotype 4.

6. A composition according to claim 3, wherein the BTV is serotype 8.

7. A composition according to claim 1, wherein the BTV is chemically inactivated.

8. A composition according to claim 7, wherein binary ethylenimine (BEA/BEI) and/or formaldehyde is used to inactivate the BTV.

9. A composition according to claim 1, which further includes an adjuvant.

10. A composition according to claim 9, wherein the adjuvant is selected from the group consisting of Montanide ISA 70, ISA 206, IMS 2214, and aluminium hydroxide and saponin.

11. A composition according to claim 1, for preventing a disease in a ruminant.

12. A composition according to claim 11, wherein the disease is Bluetongue Disease.

13. (canceled)

14. A composition according to claim 11, wherein the ruminant is selected from the group consisting of sheep, cattle, goats, and wild ungulates.

15. A composition according to claim 1, which is formulated for intramuscular or subcutaneous administration in two or more doses.

16-19. (canceled)

20. A method of producing a composition of claim 1, the method comprising the step of inactivating a live attenuated BTV.

21. A method according to claim 20, which includes an earlier step of attenuating the BTV.

22. A method for preventing a disease in ruminant, the method comprising administering to the ruminant a composition of claim 1.

23. A method according to claim 22, wherein the composition elicits an immune response in the ruminant.

24. A method according to claim 22, wherein the composition elicits antibodies specific for a BTV antigen.
